(19) United States
(12) Patent Application Publication
(10) Pub. No.: US 2021/0386730 A1
(43) Pub. Date: Dec. 16, 2021

(54) PHARMACEUTICAL FORMULATION CONTAINING GLYCOPYRROLATE AND INDACATEROL MALEATE

(71) Applicants: Cai Gu Huang, Shrewsbury, MA (US); Xiao Qian Wang, Shanghai (CN)

(72) Inventors: Cai Gu Huang, Shrewsbury, MA (US); Xiao Qian Wang, Shanghai (CN)

(73) Assignee: Cai Gu Huang, Shrewsbury, MA (US)

(21) Appl. No.: 17/342,859

(22) Filed: Jun. 9, 2021

Related U.S. Application Data
(60) Provisional application No. 63/036,469, filed on Jun. 9, 2020.

(51) Int. Cl. A61K 31/4704; A61K 31/40; A61K 9/00; A61K 47/18; A61K 47/40; A61M 11/00; A61M 15/00

(52) U.S. Cl. CPC A61K 31/4704; A61K 31/40; A61K 9/0078; A61K 9/008; A61M 15/007; A61K 47/183; A61K 47/40; A61M 11/00; A61K 47/186

(57) ABSTRACT

The present invention relates to a liquid pharmaceutical preparation and a method for administering the pharmaceutical preparation by nebulizing the pharmaceutical preparation in an inhaler. The propellant-free pharmaceutical preparation comprises: (a) glycopyrrolate and indacaterol maleate; (b) a solvent; (c) a pharmacologically acceptable solubilizing agent; (d) a pharmacologically acceptable preservative, (e) a pharmacologically acceptable stabilizer.

### PRIORITY STATEMENT

This application claims the benefit of the filing date of U.S. Provisional Patent Application No. 63/036,469, filed on Jun. 9, 2020, which is incorporated herein by reference in its entirety.

### BACKGROUND OF THE INVENTION

Glycopyrrolate, chemically (3RS)-3-[(2SR)-(2-cyclopentyl-2-hydroxy-2-penylacetyl) oxy]-1,1-dimethlypyrrolidinium bromide, has the following chemical structure:

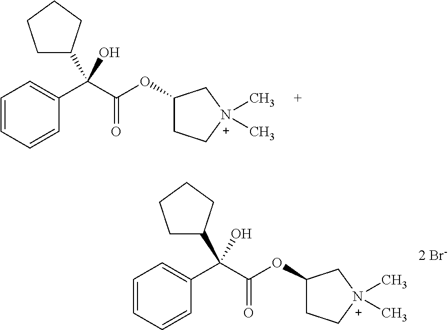 [SMILES: C[N+]1(C)CC[C@H](OC(=O)[C@](O)(c2ccccc2)C2CCCC2)C1 ; C[N+]1(C)CC[C@@H](OC(=O)[C@@](O)(c2ccccc2)C2CCCC2)C1]

Indacaterol maleate, chemically (R)-5-[2-(5,6-Diethylindan-2-ylamino)-1-hydroxyethyl]-8-hydroxy-1H-quinolin-2-one maleate, has the following chemical structure:

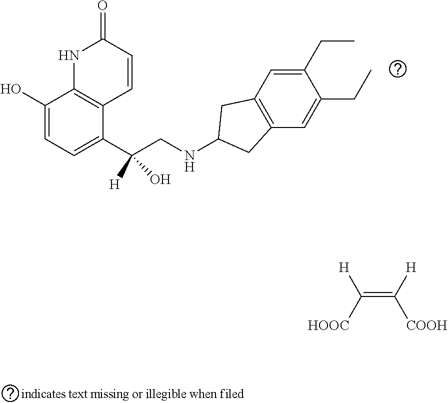 [SMILES: CCc1cc2c(cc1CC)CC(NC[C@H](O)c1ccc(O)c3[nH]c(=O)ccc13)C2 ; O=C(O)/C=C\C(=O)O]

Glycopyrrolate is a long-acting muscarinic antagonist (LAMA), which is often referred to as an anticholinergic, approved for long-term maintenance treatment of airflow obstruction in patients with chronic obstructive pulmonary disease (COPD), including chronic bronchitis and/or emphysema. In chronic obstructive pulmonary disease, acetylcholine is released to airway smooth muscle and acts reversibly through postsynaptic muscarinic receptors to mediate airway smooth contraction and mucus secretion. Inhaled anticholinergic agents can block muscarinic receptors on airway smooth muscle to inhibit bronchoconstriction.

Indacaterol is a long-acting beta-2 agonist (LABA), that works by attaching to beta-2-adrenergic receptors found in the muscles of many organs, including the airways of the lungs. When inhaled, indacaterol reaches the receptors in the airways and activates them. This causes the muscles of the airways to relax.

These two compounds have valuable pharmacological properties. Glycopyrrolate and indacaterol can provide therapeutic benefit in the treatment of asthma or chronic obstructive pulmonary disease, including chronic bronchitis and emphysema.

However, the combination of glycopyrrolate and indacaterol is currently formulated as a dry powder for inhalation. The inhalation powder is packaged in one capsule for delivery to patients only by oral inhalation using the dry powder inhalation device.

The present invention relates to a propellant-free inhalable formulation of glycopyrrolate or a solvate thereof and indacaterol or a salt or solvate thereof dissolved in water, in conjunction with inactive ingredients, preferably for administration using a soft mist or nebulization inhalation device, and the propellant-free inhalable aerosols resulting therefrom. The pharmaceutical formulations disclosed in the current invention are especially suitable for administration by soft mist inhalation or nebulization inhalation, which have much better lung depositions (typically up to 55-60%, even up to 85-95%) compared to administration of a dry powder.

The pharmaceutical formulations of the present invention are particularly suitable for administering the active substances by soft mist or nebulization inhalation, especially for treating asthma and chronic obstructive pulmonary disease.

### SUMMARY OF THE INVENTION

The present invention relates to pharmaceutical formulations of glycopyrrolate or a solvate thereof and indacaterol or pharmaceutically acceptable salt or solvate thereof which can be administered by soft mist or nebulization inhalation methods. The pharmaceutical formulations according to the invention meet high quality standards.

One aspect of the present invention is to provide an aqueous pharmaceutical formulation containing glycopyrrolate and indacaterol as a solution, which meets the high standards required to achieve optimal nebulization of the formulation using the inhalers mentioned hereinbefore. The pharmaceutical formulation has a storage time of some years, preferably at least about one year and more preferably at least about three years.

Another aspect is to provide propellant-free formulations of solutions containing glycopyrrolate and indacaterol which is nebulized under pressure using an inhaler which is preferably a soft mist or nebulization inhaler device, wherein the resulting aerosol falls reproducibly within a specified range for particle size.

Another aspect of the invention is to provide pharmaceutical formulations comprising solutions of glycopyrrolate and indacaterol and other inactive excipients which can be administered by nebulization inhalation using an ultra-sonic based or air pressure based nebulizer/inhaler. The formulation has a storage time of at least a few months or years. In one embodiment, the formulation has a storage time of at least about 1 month. In one embodiment, the formulation has a storage time of at least about 6 months. In one embodiment, the formulation has a storage time of at least about one year. In one embodiment, the formulation has a storage time of at least about three years.

Another aspect is to provide a stable pharmaceutical formulation of aqueous solutions containing glycopyrrolate and indacaterol and other excipients which can be administered by soft mist inhalation using an atomizer inhaler. In one embodiment, the formulation has substantial long term stability.

In one embodiment, the storage temperature of the formulation is from about 1° C. to about 30° C. In one embodiment, the storage temperature of the formulation is from about 15° C. to about 30° C. In one embodiment, the storage temperature of the formulation is below about 15° C. In one embodiment, the storage temperature of the formulation is from about 2° C. to about 8° C.

Another aspect of the current invention is to provide stable pharmaceutical formulations containing glycopyrrolate and indacaterol and other excipients which can be administered by nebulization inhalation using an ultrasonic, jet, or mesh nebulizer. The inventive formulation has substantial long term stability.

In one embodiment, the storage temperature of the formulation is from about 1° C. to about 30° C. In one embodiment, the storage temperature of the formulation is from about 15° C. to about 30° C. In one embodiment, the storage temperature of the formulation is below about 15° C. In one embodiment, the storage temperature of the formulation is from about 2° C. to about 8° C.

### BRIEF DESCRIPTION OF THE DRAWINGS

FIG. 1 shows a longitudinal section of an atomizer in the stressed state.

FIG. 2 shows the counter element of an atomizer.

FIG. 3 shows the particle size distribution of droplets sprayed by a soft mist inhaler according to example 3.

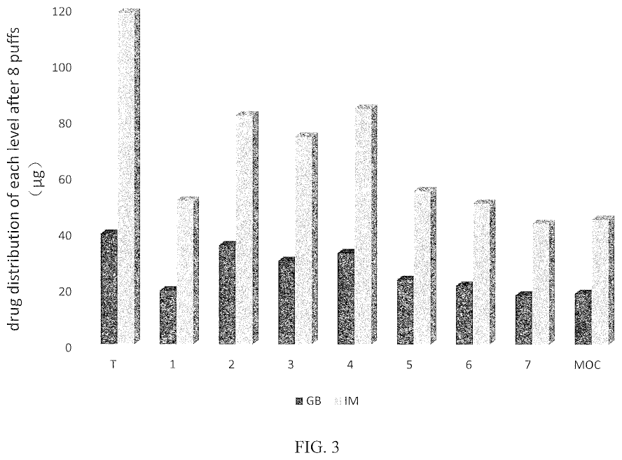

FIG. 4 shows the aerodynamic particle size distribution of glycopyrrolate (GB) and indacaterol maleate (IM) according to comparative example 3.

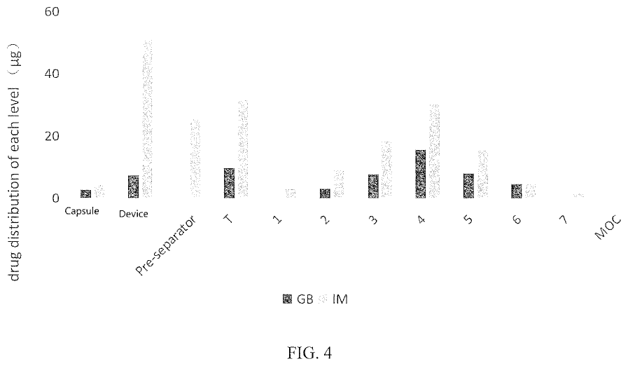

The use of identical or similar reference numerals in different figures denotes identical or similar features.

### DETAILED DESCRIPTION OF THE INVENTION

It is advantageous to use a liquid formulation without propellant gases, administered using suitable inhalers, in order to achieve better delivery of active substances in the lung. Furthermore, it is very important to increase the lung deposition of a drug delivered by inhalation.

Currently, traditional pMDI or DPI (drying powder inhalation) can only deliver about 20-30% of a drug into the lung, resulting in a significant amount of drug being deposited on the mouth and throat, which can enter the stomach and cause unwanted side effects and/or secondary absorption through the oral digestive system.

There is a need to improve inhalation drug delivery by significantly increasing lung deposition. The soft mist or nebulization inhalation device disclosed in US20190030268 can significantly increase the lung deposition of inhalable drugs.

Those inhalers can nebulize a small amount of a liquid formulation into an aerosol that is suitable for therapeutic inhalation within a few seconds. Those inhalers are particularly suitable for the liquid formulations of the invention.

In one embodiment, the soft mist or nebulization devices useful for administering the aqueous pharmaceutical formulation of the present invention are those in which an amount of less than about 70 microliters of the pharmaceutical formulation can be nebulized in one puff, so that the inhalable part of aerosol corresponds to a therapeutically effective quantity. In one embodiment, less than about 30 microliters of the pharmaceutical formulation can be nebulized in one puff, so that the inhalable part of aerosol corresponds to a therapeutically effective quantity. In one embodiment, less than about 15 microliters of the pharmaceutical formulation can be nebulized in one puff, so that the inhalable part of aerosol corresponds to a therapeutically effective quantity. In one embodiment, the average particle size of the aerosol formed from one puff is less than about 15 microns. In one embodiment, the average particle size of the aerosol formed from one puff is less than about 10 microns.

In one embodiment, the nebulization devices used to administer the pharmaceutical formulations of the present invention are those in which an amount of less than about 8 milliliters of the pharmaceutical formulation can be nebulized in one puff, so that the inhalable part of aerosol corresponds to the therapeutically effective quantity. In one embodiment, less than about 2 milliliters of the pharmaceutical formulation can be nebulized in one puff, so that the inhalable part of aerosol corresponds to the therapeutically effective quantity. In one embodiment, less than about 1 milliliter of the pharmaceutical formulation can be nebulized in one puff, so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical composition for inhalation is described in detail, for example, in US20190030268 entitled “inhalation atomizer comprising a blocking function and a counter”.

The pharmaceutical formulation is converted by the nebulizer into an aerosol destined for the lungs. The pharmaceutical solution is sprayed with the nebulizer by high pressure.

The pharmaceutical formulation is stored in a reservoir in this kind of inhaler. The formulations must not contain any ingredients which might interact with the inhaler and affect the pharmaceutical quality of the formulation or of the aerosol produced. In addition, the active substances in the pharmaceutical formulations are very stable when stored and can be administered directly.

In one embodiment, the formulations of the current invention for use with the inhaler described above contain additives, such as the disodium salt of edetic acid (sodium edetate), to reduce the incidence of spray anomalies and to stabilize the formulation. The formulations of the invention preferably have a minimum concentration of sodium edetate.

One aspect of the present invention is to provide a pharmaceutical formulation containing glycopyrrolate and indacaterol, which meets the high standards needed to achieve optimal nebulization of a solution using a soft mist inhaler. In one embodiment, the formulation has a storage time of at least a few months or years. In one embodiment, the formulation has a storage time of at least about 1 month. In one embodiment, the formulation has a storage time of at least about 6 months. In one embodiment, the formulation has a storage time of at least about one year. In one embodiment, the formulation has a storage time of at least about three years.

Another aspect of the current invention is to provide propellant-free formulations that are solutions containing glycopyrrolate and indacaterol, which can be nebulized under pressure using an inhaler, preferably a soft mist inhaler or other nebulization inhaler, such that the resulting aerosol has a particle size that falls reproducibly within a specified range.

Another aspect is to provide an aqueous pharmaceutical formulation that is a solution containing glycopyrrolate and indacaterol and other inactive excipients that can be administered by inhalation.

According to the invention, any glycopyrrolate or pharmaceutically acceptable solvate thereof and indacaterol or any pharmaceutically acceptable salt or solvate thereof may be used in the formulation. When the terms glycopyrrolate and indacaterol are used within the scope of the present invention, this is to be taken as a reference to any glycopyrrolate or pharmaceutically acceptable solvate thereof and to indacaterol or any pharmaceutically acceptable salt or solvate thereof.

Within the scope of the present invention glycopyrrolate bromide and indacaterol maleate are preferred.

In one embodiment, the active substances are selected from combinations of glycopyrrolate and indacaterol maleate.

In one embodiment, the glycopyrrolate and indacaterol maleate are dissolved in a solvent. In one embodiment, the solvent is water.

In one embodiment, a therapeutically effective dose of glycopyrrolate is about 1 μg to about 142 μg. In one embodiment, a therapeutically effective dose of glycopyrronium bromide ranges from about 1 μg to about 100 μg. In one embodiment, a therapeutically effective dose of glycopyrronium bromide ranges from about 5 μg to about 50 μg. In one embodiment, a therapeutically effective dose of glycopyrronium bromide ranges from about 10 μg to about 35 μg. In one embodiment, a therapeutically effective dose of glycopyrrolate is about 35 μg. In one embodiment, a therapeutically effective dose of indacaterol maleate ranges from about 5 μg to about 500 μg. In one embodiment, a therapeutically effective dose of indacaterol maleate is about 17 μg to about 283 μg. In one embodiment, a therapeutically effective dose of indacaterol maleate ranges from about 10 μg and 200 μg. In one embodiment, a therapeutically effective dose of indacaterol maleate ranges from about 10 μg and 80 μg. In one embodiment, a therapeutically effective dose of indacaterol maleate is about 70 μg.

The concentration of the glycopyrrolate and indacaterol in the finished pharmaceutical preparation depends on the therapeutic effects and the inhalation delivery device. In one embodiment, the concentration of glycopyrrolate in the formulation for soft mist inhalation ranges from about 8 mcg/4 ml to about 22 mg/4 ml. In one embodiment, the concentration of glycopyrrolate in the formulation for soft mist inhalation ranges from about 64 mcg/4 ml to about 12 mg/4 ml. In one embodiment, the concentration of glycopyrrolate in the formulation for soft mist inhalation ranges from about 0.252 mg/4 ml to about 5.68 mg/4 ml. In one embodiment, the concentration of indacaterol in the formulation for soft mist inhalation ranges from about 0.04 mg/4 ml to about 40 mg/4 ml. In one embodiment, the concentration of indacaterol in the formulation for soft mist inhalation ranges from about 0.8 mg/4 ml to about 20 mg/4 ml. In one embodiment, the concentration of indacaterol in the formulation for soft mist inhalation ranges from about 2 mg/4 ml to about 14 mg/4 ml.

In one embodiment, the concentration of glycopyrrolate in the formulation for nebulization inhalation ranges from about 1 mcg/ml to about 100 mcg/ml. In one embodiment, the concentration of glycopyrrolate in the formulation for nebulization inhalation ranges from about 5 mcg/ml to about 100 mcg/ml. In one embodiment, the concentration of glycopyrrolate in the formulation for nebulization inhalation ranges from about 10 mcg/ml to about 50 mcg/ml. In one embodiment, the concentration of indacaterol in the formulation for nebulization inhalation ranges from about 2 mcg/ml to about 500 mcg/ml. In one embodiment, the concentration of indacaterol in the formulation for nebulization inhalation ranges from about 10 mcg/ml to about 200 mcg/ml. In one embodiment, the concentration of indacaterol in the formulation for nebulization inhalation ranges from about 30 mcg/ml to about 100 mcg/ml.

The soft mist devices useful for administering the pharmaceutical formulation of the invention can to atomize about 10 to about 15 microliters of the pharmaceutical solution, 1 to 4 times per use, so that the inhalable part of aerosol corresponds to the therapeutically effective quantity.

In one embodiment, the formulations according to the invention include an acid or a base, as a pH adjusting agent. Suitable pH adjusting agents include, but are not limited to, hydrochloric acid, citric acid or its buffer and/or the salts thereof.

Other pH adjusting agents can be used in the present invention. In one embodiment, the pH adjusting agent is sodium hydroxide.

The pH is of the formulation is selected so as to assure suitable stability of the formulation. In one embodiment, the pH of the formulation ranges from about 2.0 to about 6.0. In one embodiment, the pH of the formulation ranges from about 3.0 to about 5.0. In one embodiment, the pH of the formulation ranges from about 3.0 to about 4.0.

In one embodiment, the formulations according to the invention include edetic acid (EDTA) or one of the known salts thereof, disodium edetate, or edetate disodium dihydrate as a stabilizer or complexing agent. In a one embodiment, the formulation contains edetic acid and/or a salt thereof.

Other comparable stabilizers or complexing agents can be included in the formulation. Examples of other stabilizers or complexing agents include, but are not limited to, citric acid, edetate disodium, and edetate disodium dihydrate.

The phrase “complexing agent,” as used herein means a molecule that is capable of entering into complex bonds. Preferably, these compounds have the effect of complexing cations. In one embodiment, the concentration of the stabilizers or complexing agents ranges from about 0.04 mg/4 ml to about 20 mg/4 ml. In one embodiment, the concentration of the stabilizers or complexing agents ranges from about 0.2 mg/4 ml to about 8 mg/4 ml. In one embodiment, the stabilizer or complexing agent is edetate disodium dihydrate in a concentration of about 0.4 mg/4 ml.

In one embodiment, all the ingredients of the formulation are present in solution.

The term “additive,” as used herein, means any pharmacologically acceptable and therapeutically useful substance which is not an active substance, but can be formulated together with the active substances in the pharmacologically suitable solvent, in order to improve the qualities of the formulation. Preferably, these substances have no appreciable pharmacological effect or, at least no undesirable pharmacological effect in the context of the desired therapy.

Suitable additives include, but are not limited to, other stabilizers; complexing agents; antioxidants; surfactants; preservatives which prolong the shelf life of the finished pharmaceutical formulation; vitamins; and/or other additives known in the art.

Preservatives protect the formulation from contamination with pathogenic bacteria. Suitable preservatives include, but are not limited to, benzalkonium chloride, benzoic acid, and sodium benzoate. In one embodiment, the formulations contain benzalkonium chloride as the only preservative. In one embodiment, the preservative is included in an amount ranging from about 0.08 mg/4 ml to about 12 mg/4 ml. In one embodiment, the preservative is benzalkonium chloride in an amount of about 0.4 mg/4 ml.

In one embodiment, the formulations include a solubility enhancing agent, such as Tween 80 or a cyclodextrin derivative. In one embodiment, the solubility enhancing agent is a cyclodextrin derivative or a salt thereof. The solubility enhancing agent improves solubility of the active ingredients and/or other excipients. In one embodiment, the solubility enhancing agent is sulfobutylether β-cyclodextrin or a salt thereof.

In one embodiment of the soft mist inhalation formulations, the formulation includes a surfactant or other solubility enhancing agent, such as Tween 80 or a cyclodextrin derivative. In one embodiment, the surfactant or other solubility enhancing agent is a cyclodextrin derivative or a salt thereof. In one embodiment, the solubility enhancing agent is sulfobutylether β-cyclodextrin. In one embodiment, the concentration of sulfobutylether β-cyclodextrin ranges from about 0.04 g/4 ml to about 1.6 g/4 ml. In one embodiment, the solubility enhancing agent is sulfobutylether β-cyclodextrin in a concentration of about 0.8 g/4 ml.

Another aspect of the current invention is to provide a stable pharmaceutical formulation that is a solution comprising glycopyrrolate and indacaterol and other excipients which can be administered by soft mist inhalation using an atomizer inhaler. In one embodiment, the formulation has substantial long term stability.

In one embodiment, the storage temperature of the formulation is from about 1° C. to about 30° C. In one embodiment, the storage temperature of the formulation is from about 15° C. to about 30° C. In one embodiment, the storage temperature of the formulation is below about 15° C. In one embodiment, the storage temperature of the formulation is from about 2° C. to about 8° C.

Another aspect of the current invention is to provide a pharmaceutical formulation that is a solutions comprising glycopyrrolate and indacaterol and other inactive excipients which can be administered by nebulization inhalation using an ultra-sonic based or air pressure based nebulizer/inhaler. In one embodiment, the formulation has substantial long term stability.

In one embodiment, the formulations storage temperature is from about 1° C. to about 30° C. In one embodiment, the formulations storage temperature is from about 15° C. to about 30° C. In one embodiment, the formulations storage temperature is below 15° C. In one embodiment, the formulations storage temperature is from about 2° C. to about 8° C.

In one embodiment, the formulations include sodium chloride. In one embodiment, the concentration of sodium chloride ranges from about 0.1 g/100 ml to about 0.9 g/100 ml.

In one embodiment, the concentration of glycopyrrolate in the formulation for nebulization inhalation ranges from about 1 mcg/ml to about 100 mcg/ml. In one embodiment, the concentration of glycopyrrolate in the formulation for nebulization inhalation ranges from about between 5 mcg/ml to about 100 mcg/ml. In one embodiment, the concentration of glycopyrrolate in the formulation for nebulization inhalation ranges from about 10 mcg/ml to about 50 mcg/ml. In one embodiment, the concentration of indacaterol in the formulation for nebulization inhalation ranges from about 2 mcg/ml to about 500 mcg/ml. In one embodiment, the concentration of indacaterol in the formulation for nebulization inhalation ranges from about 10 mcg/ml to about 200 mcg/ml. In one embodiment, the concentration of indacaterol in the formulation for nebulization inhalation ranges from about 30 mcg/ml to about 100 mcg/ml.

In one embodiment, the formulations for nebulization inhalation according to the invention include a surfactant or other solubility enhancing agent, such as Tween 80 or a cyclodextrin derivative. In one embodiment, the surfactant or other solubility enhancing agent is a cyclodextrin derivative or a salt thereof. In one embodiment, the surfactant or other solubility enhancing agent is sulfobutylether β-cyclodextrin. In one embodiment, the surfactant or other solubility enhancing agent is sulfobutylether β-cyclodextrin in an amount ranging from about 5 mg/ml to about 0.4 g/ml. In one embodiment, the sulfobutylether β-cyclodextrin is in an amount of about 0.2 g/ml.

It has been found that sulfobutylether β-cyclodextrin not only enhances solubility, but advantageously improves the stability of the active ingredients.

Another aspect of the invention is to provide a stable pharmaceutical formulation for nebulization comprising glycopyrrolate and indacaterol and other excipients which can be administered by soft mist inhalation using an atomizer inhaler. In one embodiment, the formulation has substantial long-term stability.

In one embodiment, the storage temperature of the formulation is from about 1° C. to about 30° C. In one embodiment, the storage temperature of the formulation is from about 15° C. to about 30° C. In one embodiment, the storage temperature of the formulation is below about 15° C. In one embodiment, the storage temperature of the formulation is from about 2° C. to about 8° C.

The pH of the formulation influences the stability of the glycopyrrolate and indacaterol in the nebulization formulation. The pH can be adjusted to the desired value by adding an acid, e.g., HCl, or by adding a base, e.g., NaOH.

In one embodiment, the pH of the nebulization formulation ranges from about 3 to about 6. In one embodiment, the pH of the nebulization formulation ranges from about 3 to about 5. In one embodiment, the pH of the nebulization formulation ranges from about 3 to about 4.

In one embodiment, the invention is directed to a liquid, propellant-free pharmaceutical formulation comprising:
an aqueous solution of:
   (a) glycopyrrolate in an amount of about 0.2 mg/100 mL to about 550 mg/100 mL,
   (b) indacaterol maleate in an amount of about 0.34 mg/100 mL to about 1000 mg/100 mL,
   (c) sulfobutylether β-cyclodextrin (SBECD) in an amount of about 1 g/100 mL to about 40 g/100 mL,
   (d) 50% benzalkonium chloride aqueous solution in an amount of about 2 mg/100 mL to about 300 mg/100 mL, and
   (e) edetate disodium dihydrate in an amount of about 1 mg/100 mL to about 500 mg/100 mL,
wherein the pharmaceutical preparation has a pH ranging from about 3.0 to about 4.0.

Because the ISM of the self-made formulation is much higher than that of the original research, in order to be consistent with the original research, it is considered that the effective dose of glycopyrrolate and indacaterol maleate can be reduced.

In one embodiment, the invention is directed to a method for preparing a pharmaceutical formulation comprising a solution of indacaterol maleate in water comprising: (i) adding about 3.4 mg to about 10 g of indacaterol maleate to about 100 g of water substantially free of other solvents to provide a suspension and (ii) heating the suspension at a temperature ranging from about 50 to about 90° C. with stirring until the indacaterol dissolves.

In one embodiment, the invention is directed to a method of preparing a pharmaceutical formulation comprising a solution of indacaterol maleate in water comprising: (i) combining about 10 to about 400 g of sulfobutylether β-cyclodextrin (SBECD), about 10 mg to about 5000 mg of EDTA, and about 20 to about 3000 mg of 50% benzalkonium chloride aqueous solution and about 50 g of water to provide a first solution; (ii) adding about 3.4 mg to about 10 g of indacaterol maleate to about 50 g of water to provide a first suspension, then adding the first suspension to the first solution to provide a second suspension; (iii) heating the second suspension at a temperature of about 50 to about 90° C. with stirring until the indacaterol maleate dissolves to provide a second solution; (iv) adding about 20 mg to about 3000 mg of glycopyrronium bromide to the second solution to provide a mixture, and (v) stirring the mixture until the glycopyrronium bromide dissolves.

The nebulization formulations according to the present invention can be filled into canisters to provide a highly stable formulation for use in the nebulization device. The formulations exhibit substantially no particle growth, change of morphology, or precipitation. There also is no, or substantially no, problem of suspended particles being deposited on the surface of either the canister or the valves, so that the formulations can be discharged from the nebulization device with high dose uniformity. Suitable nebulizers include, but are not limited to, an ultrasonic nebulizer; a jet nebulizer; a mesh nebulizer, such as Pari eFlow nebulization inhaler, or other commercially available ultrasonic nebulizer, jet nebulizer or mesh nebulizer.

In one embodiment, the inhalation device is a soft mist inhaler. To produce the aerosols, the pharmaceutical soft mist formulations containing glycopyrrolate and indacaterol is preferably administered using in an inhaler of the kind described herein. Here, we once again expressly mention the patent documents described hereinbefore, to which reference is hereby made.

A soft mist inhaler device of this kind for the propellant-free administration of a metered amount of a liquid pharmaceutical composition for inhalation is described in detail, for example, in US20190030268 entitled “inhalation atomizer comprising a blocking function and a counter”.

The pharmaceutical formulation is a solution that is converted by the nebulizer into an aerosol destined for the lungs. The nebulizer uses high pressure to spray the pharmaceutical solution.

The soft mist inhalable device can be carried anywhere by the patient, since it has a cylindrical shape and handy size of about 8 cm to 18 cm long, and about 2.5 cm to 5 cm wide. The nebulizer sprays a defined volume of the pharmaceutical formulation through small nozzles at high pressures, so as to produce inhalable aerosols.

In one embodiment, the delivery device comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

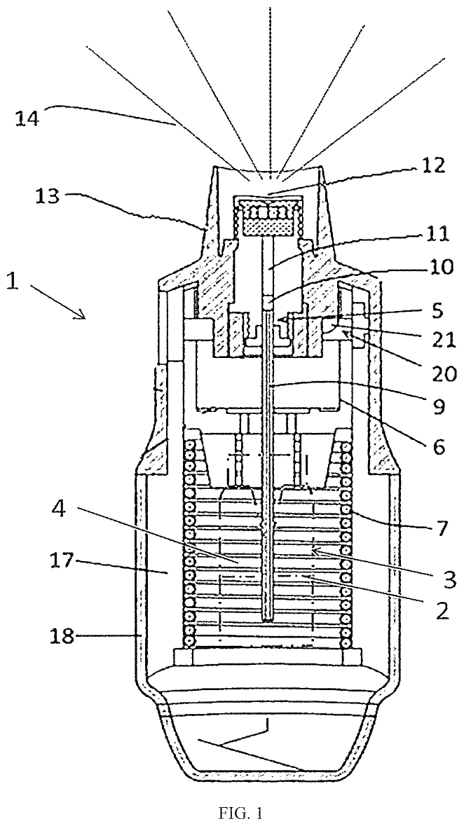

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and propellant-free.

FIG. 1 shows a longitudinal section through the atomizer in a stressed state.

For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predestined time gaps, according to how serious the illness affects the patient.

In an embodiment, the atomizer 1 described above has substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 comprising the block function and the counter described above is in the vessel 3 to provide, e.g., up to 200 doses. A classical vessel 3 has a volume of 2 to 10 ml. A pressure generator 5 in the atomizer 1 is used to deliver and atomize the fluid 2 in a predetermined dosage amount. Therefore, the fluid 2 can be released and sprayed in individual doses, specifically from 5 to 30 microliter.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 could be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and the non-return valve 10.

In one embodiment, after releasing the holder 6, the stress is eased. During this process, the delivering tube 9 and closed non-return valve 10 are shifted back upward by releasing the drive spring 7. Consequently, the fluid 2 is under pressure in the pressure room 11. Then the fluid 2 is pushed through the nozzle 12 and atomized into an aerosol 14 by the pressure. A patient could inhale the aerosol 14 through the mouthpiece 13, while the air is sucked into the mouthpiece 13 through air inlets 15.

The inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In one embodiment, the inhalation atomizer 1 described above has the lower shell 18, which carries the inside part 17, being rotatable relative to the upper shell 16. As a result of rotation and engagement between the upper unit 17 and the holder 6, through a gear 20, the holder 6 is axially moved counter to the force of the drive spring 7 and the drive spring 7 is stressed.

In an embodiment, in the stressed state, the vessel 3 is shifted downwards and reaches to a final position, which is demonstrated in the FIG. 1. The drive spring 7 is stressed under this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting in here. While the major shifting occurs, the non-return valve 10 is closed and the fluid 2 is under pressure in the pressure room 11 by the delivering tube 9, and fluid 2 is pushed out and atomized by the pressure.

In an embodiment, the inhalation atomizer 1 described above may have a clamping function. During clamping, the vessel 3 preferably performs a lifting shift for withdrawal of fluid 2 during the atomizing process. The gear 20 has sliding surfaces 21 on the upper shell 16 and/or on the holder 6, which makes holder 6 move axially when the holder 6 is rotated relative to the upper shell 16.

In an embodiment, the holder 6 is not blocked for too long and can perform the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite shift axially.

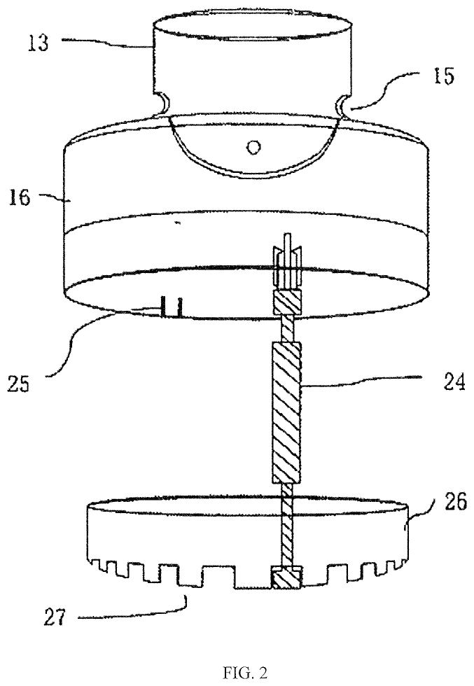

In an embodiment, the atomizer 1 preferably includes a counter element shown in FIG. 2. The counter element has a worm 24 and a counter ring 26. Preferably, the counter ring 26 is circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once a predetermined number of actuations is achieved, Protrusion A and Protrusion B will encounter with each other and hence the counter will be prevented from further rotation. Therefore, the atomizer is blocked and stopped from further use. The number of actuations of the device can be counted by the counter.

Atomization devices include, but are not limited to, soft mist inhalers, ultrasonic atomizers, air compression atomizers, and mesh-based atomizers.

The soft mist inhalers use pressure to eject a metered dose drug solution. Two high-speed jets are formed, and the two jets collide with each other to form droplets with smaller particles.

With an ultrasonic atomizer, the oscillation signal of the main circuit board is amplified by a high-power triode and transmitted to the ultrasonic wafer. The ultrasonic wafer converts electrical energy into ultrasonic energy. The ultrasonic energy can atomize the water-soluble drug into tiny mist particles ranging in size from about 1 um to about 5 um at normal temperature. With the help of an internal fan, the medicine particles are ejected.

An air compression atomizer is mainly composed of a compressed air source and an atomizer. The compressed gas is suddenly decompressed after passing through a narrow opening at high speed and a negative pressure is generated locally so that the solution of the active substance is sucked out from the container because of a siphon effect. When subject to high-speed air flow, the solution of active substance is broken into small aerosol particles by collision.

Mesh based atomizers contains a stainless-steel mesh covered with micropores having a diameter of about 3 μm. The number of micropores exceeds 1,000. The mesh is conical, with the cone bottom facing the liquid surface. Under the action of pressure, the vibration frequency of the mesh is about 130 KHz. The high vibration frequency breaks the surface tension of the drug solution contacted with the mesh, and produces a low-speed aerosol.

### EXAMPLES

Materials and reagents:
50% benzalkonium chloride aqueous solution purchased from Merck,
Edetate disodium dehydrate purchased from Merck,
Sodium hydroxide purchased from Titan reagents,
Hydrochloric acid purchased from Titan reagents,
Citric acid purchased from Merck,
Sodium chloride purchased from Titan reagents,
Glycopyrrolate purchased from Nanjing Daqin Pharma Co., Ltd,
Indacaterol maleate purchased from Nanchang Anovent Pharma Co., Ltd.

### Example 1

A formulation of a soft mist inhalation solution (Sample I) was prepared as follows:

50% benzalkonium chloride aqueous solution and Edetate Disodium Dihydrate and SBECD according to the amounts in Table 1, were dissolved in 90 ml of purified water. Glycopyrrolate and indacaterol maleate according to the amounts in Table 1 were added to the solution, and the resulting mixture sonicated until the components were completely dissolved. The solution was then adjusted to the target pH with hydrochloric acid or sodium hydroxide. Finally, purified water was added to provide a final volume of 100 ml.

TABLE 1
Ingredients of Sample I

| | Quantity in unit dosage | | Quantity for | |
|---|---|---|---|---|
| Ingredients | form (for 60 doses) | | preparation | |
| Glycopyrrolate | 2.84 | mg | 71 | mg |
| Indacaterol Maleate | 6.44 | mg | 161 | mg |
| Sulfobutylether β-Cyclodextrin | 0.2 | g | 5 | g |
| (SBECD) | | | | |
| 50% benzalkonium chloride | 0.8 | mg | 20 | mg |
| aqueous solution | | | | |
| Edetate disodium dihydrate | 0.4 | mg | 10 | mg |

| | | |
|---|---|---|
| Hydrochloride Acid | To pH 3.0 | To pH 3.0 |
| Purified water | Added to 4 ml | Added to 100 ml |

### Example 2

Aerodynamic Particle Size Distribution of the Soft Mist Inhalation Solution (Sample I from Example 1):

The soft mist inhalation solution of Example 1 (i.e., sample I) was sprayed using a soft mist inhaler. The aerodynamic particle size distribution of the resulting droplets were measured using a Next Generation Impactor (NGI). The Next Generation Impactor was operated at a flow rate of 30 L/min to determine the particle size distribution. For each of the experiments, the impactor collection stages were coated with a silicone oil. The particle size distribution is expressed in terms of mass median aerodynamic diameter (MMAD) and Geometric Standard Deviation (GSD). The results are provided in Table 2. The results show that the MMAD of indacaterol maleate and glycopyrrolate was less than 10 μm and that the GSD of indacaterol maleate and glycopyrrolate was less than 5% (Table 2).

TABLE 2
Aerodynamic Particle Size Distribution of the Soft Mist Inhalation
Solution (Sample I in Example 1)

| Particle size parameter | Indacaterol Maleate | Glycopyrrolate |
|---|---|---|
| MMAD (μm) | 3.18 | 3.15 |
| GSD (%) | 3.5 | 3.7 |

### Example 3

Sample I from Example 1 was sprayed using a soft mist inhalation device. A Malvern Spraytec (STP5313) was used to measure the particle size of the droplets. The results are shown in Table 3.

TABLE 3
Droplet Particle Size Distribution of Sample I of Example 1 Using a
Soft Mist Inhaler

| Test time | Dv (10) μm | Dv (50) μm | Dv (90) μm |
|---|---|---|---|
| 1 | 2.9 | 5.6 | 10.3 |
| 2 | 2.8 | 5.4 | 9.9 |
| 3 | 2.7 | 5.3 | 9.5 |

### Example 4

A formulation of a nebulization inhalation solution (sample II) was prepared as follows:

Sodium chloride and SBECD according to the amounts in Table 4, were dissolved in 90 ml of purified water. Indacaterol maleate and glycopyrrolate according to the amounts in Table 4 were added to the solution, and the resulting mixture sonicated until the components completely dissolved. The solution was then adjusted to the target pH with hydrochloric acid or sodium hydroxide. Finally, purified water was added to final volume of 100 ml.

TABLE 4
Ingredients of Sample II

| | Quantity in unit dosage | | Quantity for | |
|---|---|---|---|---|
| Ingredients | form (for single use) | | preparation | |
| Glycopyrrolate | 0.04 | mg | 4 | mg |
| Indacaterol Maleate | 0.09 | mg | 9 | mg |
| Sulfobutylether β-Cyclodextrin | 10 | mg | 1 | g |
| (SBECD) | | | | |
| Sodium chloride | 7.5 | mg | 750 | mg |

| | | |
|---|---|---|
| Hydrochloric acid or sodium | To pH 4.0 | To pH 4.0 |
| hydroxide | | |
| Purified water | Added to 1 ml | Added to 100 ml |

### Example 5

Solubility of indacaterol maleate and glycopyrrolate:

A solution of indacaterol maleate and glycopyrrolate formulation is a transparent solution. In a formulation it is necessary to be sure that the active pharmaceutical ingredients (API) are dissolved completely. Provided below is solubility data for indacaterol maleate and glycopyrrolate.

TABLE 5
Glycopyrrolate Solubility Data

| Solvent | Solubility (mg/ml) |
|---|---|
| pH = 2 hydrochloric acid solution | 122.77 mg/ml |
| pH = 4 hydrochloric acid solution | 122.63 mg/ml |
| pH = 6 hydrochloric acid solution | 98.43 mg/ml |

TABLE 6
Indacaterol Maleate Solubility Data

| Solvent (pH conditioner is 1N | |
|---|---|
| hydrochloric acid) | Solubility (mg/100 ml) |
| Purified water | 29.95 mg/100 ml |
| pH = 3 Purified water | 31.21 mg/100 ml |
| pH = 3.5 Purified water | 29.97 mg/100 ml |
| pH = 4 Purified water | 30.82 mg/100 ml |
| pH = 4.5 Purified water | 30.32 mg/100 ml |
| pH = 5 Purified water | 28.98 mg/100 ml |
| 0.02% tween-80 | 29.52 mg/100 ml |
| pH = 3 0.02% tween-80 | 32.17 mg/100 ml |
| pH = 3.5 0.02% tween-80 | 30.8 mg/100 ml |
| pH = 4 0.02% tween-80 | 30.91 mg/100 ml |
| pH = 4.5 0.02% tween-80 | 30.41 mg/100 ml |
| pH = 5 0.02% tween-80 | 30.84 mg/100 ml |
| pH = 4 5% SBECD solution | 316 mg/100 ml |
| pH = 3 5% SBECD solution | 539.5 mg/100 ml |
| pH = 4 1% SBECD solution | 161.866 mg/100 ml |
| pH = 4 2% SBECD solution | 211.4 mg/100 ml |
| pH = 4 0.5% SBECD solution | 55.523 mg/100 ml |
| pH = 3 2.5% SBECD solution | 351.46 mg/100 ml |

0.02% tween-80: 2 g tween-80 dissolved in 98 g purified water.

pH=3 0.02% tween-80: 2 g tween-80 dissolved in 98 g purified water, adjust pH to 3 with hydrochloric acid. Other solutions were similarly prepared. The percentages mentioned above are percentages by weight.

Conclusion: In purified water, indacaterol maleate (IM) is not sufficiently soluble to prepare a solution for soft mist inhalation. When tween 80 was added, the solubility of IM was also insufficient. When cyclodextrin was added, the solubility of IM improved significantly, such that a concentration sufficient for preparing a solution for soft mist inhalation can be achieved.

### Example 6

Influence of pH on stability:

The stability of a glycopyrrolate (referred as GB) and indacaterol maleate (referred as IM) solution is highly dependent on pH. Four samples, were prepared at pH values of 3, 4, 4.5, and unadjusted pH according to the following procedure: 50% benzalkonium chloride aqueous solution, SBECD, and Edetate Disodium Dihydrate according to the amounts in Table 7 were dissolved in 28 g of purified water and the resulting solution adjusted to the target pH with 1 N HCl. Glycopyrrolate and indacaterol maleate according to the amounts provided in Table 7 were then added to the solution, and the resulting mixture sonicated until the components completely dissolved. Finally, purified water was added to provide a final weight of 30.21 g.

TABLE 7
Formulations of GB and IM at Different pH Values

| Ingredients | Sample 1 | | Sample 2 | | Sample 3 | | Sample 4 | |
|---|---|---|---|---|---|---|---|---|
| GB | 21.3 | mg | 21.3 | mg | 21.3 | mg | 21.3 | mg |
| IM | 48.3 | mg | 48.3 | mg | 48.3 | mg | 48.3 | mg |
| SBECD | 0.75 | g | 0.75 | g | 0.75 | g | 0.75 | g |
| EDTA | 3 | mg | 3 | mg | 3 | mg | 3 | mg |
| 50% BAC | 6 | mg | 6 | mg | 6 | mg | 6 | mg |

| | | | | |
|---|---|---|---|---|
| 1N HCl | 3 | 4 | 4.5 | Don't adjust pH |
| purified water | Added to | Added to | Added to | Added to |
| | 30.21 g | 30.21 g | 30.21 g | 30.21 g |

The solution was packaged into small brown bottles, capped with a lid, the bottle sealed with a film, and placed in a 60° C. oven. On days 0, 5, and 10, samples were analyzed for impurities.

Analysis method:
Mobile phase A: Weigh 3.64 g NaH₂PO₄, dissolved in 1 L water, pH 3.5.
Mobile phase B: Acetonitrile (ACN)
Column: ODS-3-C18, 5 μm, 150×4.6 mm
Flow rate: 1.0 mL/min
Injection Volume: 10 μL
Run Time: 66 minutes
Detection Wavelength: 210 nm
Gradient elution:

| Time(min) | Mobile phase A (%) | Mobile phase B (%) |
|---|---|---|
| 0 | 90 | 10 |
| 15 | 75 | 25 |
| 30 | 65 | 35 |
| 45 | 45 | 65 |
| 55 | 30 | 70 |
| 56 | 90 | 10 |
| 66 | 90 | 10 |

The results are provided in the Table 8.

TABLE 8
Stability of GB and IM at Different pH Values

| | | | Sample 1 | Sample 2 | Sample 3 | Sample 4 |
|---|---|---|---|---|---|---|
| 0 day | character | | Colorless | Colorless | Colorless | Colorless |
| | | | clear liquid | clear liquid | clear liquid | clear liquid |
| | impurity | Maximum | 0.22 | 0.22 | 0.22 | 0.21 |
| | % | impurity | | | | |
| | | Total | 0.31 | 0.29 | 0.30 | 0.29 |
| | | impurity | | | | |
| 60° C. | character | | Colorless | Colorless | Colorless | Colorless |
| 5 days | | | clear liquid | clear liquid | clear liquid | clear liquid |
| | impurity | Maximum | 0.25 | 0.24 | 0.23 | 0.2 |
| | % | impurity | | | | |
| | | Total | 0.36 | 0.34 | 0.36 | 0.31 |
| | | impurity | | | | |
| 60° C. | character | | Colorless | Colorless | Colorless | Colorless |
| 10 days | | | clear liquid | clear liquid | clear liquid | clear liquid |
| | impurity | Maximum | 0.24 | 0.23 | 0.23 | 0.23 |
| | % | impurity | | | | |
| | | Total | 0.34 | 0.36 | 0.41 | 0.43 |
| | | impurity | | | | |

The above experiment shows that the stability of GB and IM in solution is dependent on pH. As can be seen from Table 8, at pH 3 to 4.5 solutions of GB and IM are stable and have the best stability at pH 3.0-4.0.

### Example 7

Influence of EDTA on Stability:

The stability of glycopyrrolate and indacaterol maleate in solution is highly dependent on EDTA. Four samples were prepared having EDTA concentration of 9 mg/100 ml, 10 mg/100 ml, 11 mg/100 ml and no added EDTA were prepared as follows: 50% benzalkonium chloride aqueous solution, SBECD, and Edetate Disodium Dihydrate according to the amounts in Table 9 were dissolved in 95 g of purified water and the resulting solution adjusted to the target pH with 1 N HCl. Glycopyrrolate and indacaterol maleate according to the amounts provided in Table 9 were added to the solution, and the resulting mixture was sonicated until the components completely dissolved. Finally, purified water was added to provide a final weight of 100.72 g.

TABLE 9
Formulation of GB and IM at Different EDTA Concentrations

| Ingredients | Sample 5 | | Sample 6 | | Sample 7 | | Sample 8 | |
|---|---|---|---|---|---|---|---|---|
| GB | 71 | mg | 71 | mg | 71 | mg | 71 | mg |
| IM | 161 | mg | 161 | mg | 161 | mg | 161 | mg |
| SBECD | 2.5 | g | 2.5 | g | 2.5 | g | 2.5 | g |
| EDTA | 9 | mg | 10 | mg | 11 | mg | unadd | |
| 50% BAC | 20 | mg | 20 | mg | 20 | mg | 20 | mg |
| 1N HCl | Adjust to | | Adjust to | | Adjust to | | Adjust to | |
| | pH 3 | | pH 3 | | pH 3 | | pH 3 | |
| purified water | 100.72 | g | 100.72 | g | 100.72 | g | 100.72 | g |

The results are provided in the Table 10.

TABLE 10
Stability of GB and IM at Different EDTA Concentrations

| | | | Sample 5 | Sample 6 | Sample 7 | Sample 8 |
|---|---|---|---|---|---|---|
| 0 day | character | | Colorless | Colorless | Colorless | Colorless |
| | | | clear liquid | clear liquid | clear liquid | clear liquid |
| | impurity | Maximum | 0.22 | 0.22 | 0.21 | 0.22 |
| | % | impurity | | | | |
| | | Total | 0.30 | 0.30 | 0.30 | 0.30 |
| | | impurity | | | | |
| 60° C. | character | | Colorless | Colorless | Colorless | Colorless |
| 5 days | | | clear liquid | clear liquid | clear liquid | clear liquid |
| | impurity | Maximum | 0.23 | 0.23 | 0.24 | 0.23 |
| | % | impurity | | | | |
| | | Total | 0.36 | 0.38 | 0.45 | 0.96 |
| | | impurity | | | | |
| 60° C. | character | | Colorless | Colorless | Colorless | Colorless |
| 10 days | | | clear liquid | clear liquid | clear liquid | clear liquid |
| | impurity | Maximum | 0.23 | 0.23 | 0.24 | 0.23 |
| | % | impurity | | | | |
| | | Total | 0.41 | 0.42 | 0.41 | 1.45 |
| | | impurity | | | | |

The above experiment shows that the stability of GB and IM in solution is dependent on ETDA. As can be seen from Table 10, solutions having an EDTA concentration of about 9-11 mg/100 ml are stable.

### Example 8

Stability:

50% benzalkonium chloride aqueous solution, SBECD, and Edetate Disodium Dihydrate according to the amounts in Table 11 or Table 12 were dissolved in 95 g of purified water, and the resulting solution adjusted to the target pH with 1 N HCl. Glycopyrrolate and indacaterol maleate according to the amounts provided in Table 11 or Table 12 were added to the solution and the resulting mixture was sonicated until the components completely dissolved. Finally, purified water was added to provide a final weight of 101.8 g.

TABLE 11
Ingredient of Sample 9-12 of an Inhalation Solution

| Ingredient | Sample 9 | | Sample 10 | | Sample 11 | | Sample 12 | |
|---|---|---|---|---|---|---|---|---|
| IM | 161 | mg | 161 | mg | 161 | mg | 161 | mg |
| GB | 71 | mg | 71 | mg | 71 | mg | 71 | mg |
| SBECD | 5 | g | 5 | g | 5 | g | 5 | g |
| 50% BAC | 20 | mg | 20 | mg | 20 | mg | 20 | mg |
| EDTA | 10 | mg | 10 | mg | 10 | mg | 10 | mg |
| 1N HCl | Adjust to | | Adjust to | | Adjust to | | Adjust to | |
| | pH 3.0 | | pH 3.3 | | pH 3.6 | | pH 3.9 | |
| Purified water | 101.8 | g | 101.8 | g | 101.8 | g | 101.8 | g |

TABLE 12
Ingredient of Samples 13-14 of an Inhalation Solution

| Ingredients | Sample 13 | | Sample 14 | |
|---|---|---|---|---|
| IM | 320 | mg | 320 | mg |
| GB | 160 | mg | 160 | mg |
| SBECD | 5 | g | 5 | g |
| 50% BAC | 20 | mg | 20 | mg |
| EDTA | 10 | mg | 10 | mg |
| 1N HCl | Adjust to pH 3.0 | | Adjust to pH 3.6 | |
| Purified water | 101.8 | g | 101.8 | g |

Cyclodextrin affects the density of the solution. Accordingly, we measured the density of solutions having different cyclodextrin concentration as follows:

Sample 9-12, 13, and 14 contained 5% cyclodextrin (SBECD) and the solution density was 1.0179 g/ml.

The density was measured at a temperature of 20° C.

The solutions were filled into LDPE containers, sealed with aluminum foil, and stored at 40° C.±2° C./75%±5% RH and the stability monitored. The results are provided in Tables 13 and 14.

TABLE 13
Stability of Samples 9-12 (Condition: 40° C. ± 2° C./75% ± 5% RH)

| | | | Sample | Sample | Sample | Sample |
|---|---|---|---|---|---|---|
| | | | 9 | 10 | 11 | 12 |
| | | | pH: 3 | pH: 3.3 | pH: 3.6 | pH: 3.9 |
| 0 day | character | | Colorless clear liquid | | | |
| | pH | | 3.06 | 3.34 | 3.67 | 3.94 |
| | Content (%) | GB | 103.18 | 102.14 | 101.59 | 100.97 |
| | | IM | 102.9 | 101.94 | 101.2 | 103.97 |
| | | 50% BAC | 106.65 | 107.38 | 103.05 | 105.75 |
| | | EDTA | 99.99 | 99.11 | 99.38 | 101.95 |
| | impurity % | Maximum | 0.15 | 0.15 | 0.15 | 0.16 |
| | | impurity | | | | |
| | | Total | 0.22 | 0.21 | 0.2 | 0.21 |
| | | impurity | | | | |
| 40° C. | character | | Colorless clear liquid | | | |
| 1 month | pH | | 3 | 3.32 | 3.64 | 3.92 |
| | Content (%) | GB | 101.3 | 101.65 | 102 | 101.61 |
| | | IM | 100.95 | 101.26 | 101.59 | 104.52 |
| | | 50% BAC | 103.6 | 102.78 | 105 | 104.8 |
| | | EDTA | 100.57 | 100.03 | 99.01 | 98.91 |
| | impurity % | Maximum | 0.22 | 0.17 | 0.18 | 0.19 |
| | | impurity | | | | |
| | | Total | 0.29 | 0.24 | 0.23 | 0.24 |
| | | impurity | | | | |
| 40° C. | character | | Colorless clear liquid | | | |
| 3months | pH | | 3.01 | 3.33 | 3.62 | 3.91 |
| | Content (%) | GB | 102.32 | 102.26 | 103.22 | 102.7 |
| | | IM | 101.42 | 101.25 | 101.79 | 104.47 |
| | | 50% BAC | 100.79 | 100.68 | 100.44 | 101.19 |
| | | EDTA | 103.11 | 101.96 | 102.54 | 104.03 |
| | impurity % | Maximum | 0.21 | 0.21 | 0.21 | 0.22 |
| | | impurity | | | | |
| | | Total | 0.28 | 0.31 | 0.29 | 0.29 |
| | | impurity | | | | |

TABLE 14
Stability of Sample 13-14 (Condition: 40° C. ± 2° C./75% ± 5% RH)

| | | | Sample 13 | Sample 14 |
|---|---|---|---|---|
| | | | pH: 3 | pH: 3.6 |
| 0 day | character | | Colorless clear liquid | |
| | pH | | 3.06 | 3.62 |
| | Content | GB | 99.42 | 99.30 |
| | (%) | IM | 99.31 | 99.59 |
| | | 50% BAC | 102.60 | 101.60 |
| | | EDTA | 101.80 | 99.90 |
| | impurity % | Maximum | 0.21 | 0.14 |
| | | impurity | | |
| | | Total | 0.35 | 0.24 |
| | | impurity | | |
| 40° C. | character | | Colorless clear liquid | |
| 1 month | pH | | 3.02 | 3.61 |
| | Content | GB | 101.22 | 98.84 |
| | (%) | IM | 101.36 | 98.93 |
| | | 50% BAC | 104.10 | 103.50 |
| | | EDTA | 102.40 | 100.00 |
| | impurity % | Maximum | 0.14 | 0.14 |
| | | impurity | | |
| | | Total | 0.24 | 0.14 |
| | | impurity | | |
| 40° C. | character | | Colorless clear liquid | |
| 3 months | pH | | 3.02 | 3.6 |
| | Content | GB | 98.35 | 101.89 |
| | (%) | IM | 98.56 | 101.81 |
| | | 50% BAC | 108.10 | 109.3 |
| | | EDTA | 101.70 | 101.00 |
| | impurity % | Maximum | 0.14 | 0.13 |
| | | impurity | | |
| | | Total | 0.14 | 0.13 |
| | | impurity | | |
| 40° C. | character | | Colorless clear liquid | |
| 6 months | pH | | 3.01 | 3.61 |
| | Content | GB | 102.81 | 103.89 |
| | (%) | IM | 102.53 | 103.62 |
| | | 50% BAC | 105.10 | 105.20 |
| | | EDTA | 103.40 | 100.40 |
| | impurity % | Maximum | 0.13 | 0.14 |
| | | impurity | | |
| | | Total | 0.29 | 0.23 |
| | | impurity | | |

As can been seen from Tables 13 and 14, at pH 3.0 to 3.9 solutions of GB and IM are most stable. Under the condition of pH 3 to 3.9, solutions of GB and IM are stable for 6 months at 40° C.±2° C./75%±5% RH.

### Comparative Example 1

Indacaterol maleate and glycopyrrolate according to the amounts in Table 15, were added to 90 ml of purified water and the resulting mixture sonicated until the components completely dissolved. The solution was adjusted to the target pH with hydrochloric acid or sodium hydroxide. Finally, purified water was added to a final volume of 100 ml.

TABLE 15
Contents of Comparative Example 1

| Ingredients | Comparative Example 1 |
|---|---|
| Glycopyrrolate | 2 mg |
| Indacaterol Maleate | 4.5 mg |
| Hydrochloric acid or sodium | To pH 3.0 |
| hydroxide | |
| Purified water | Added to 100 ml |

### Comparative Example 2

The thermal stability at 60° C. of Comparative Example 1 is provided below in Table 16:

TABLE 16
Thermal Stability at 60° C. of Comparative Example 1

| | 0 d | 10 d |
|---|---|---|
| Maximum impurity | 0.03% | 0.14% |

### Comparative Example 3

Aerodynamic Particle Size Distribution of a dry powder for inhalation

The aerodynamic particle size distribution of one capsule of Ultibro (dry powder inhalation) from Novartis was measured using a Next Generation Impactor (NGI). The Next Generation Impactor operated at a flow rate of 30 L/min was used to determine the particle size distribution. For each of the impactor experiments, the impactor collection stages were coated with a silicone oil. The particle size distribution is expressed in terms of mass median aerodynamic diameter (MMAD) and Geometric Standard Deviation (GSD). The results showed that the MMAD of indacaterol maleate and glycopyrrolate was less than 5 μm and that the GSD of indacaterol maleate and glycopyrrolate was less than 5% (Table 17).

TABLE 17
Aerodynamic Particle Size Distribution of
Ultibro (Dry Powder Inhalation) from Novartis

| Particle size parameter | Indacaterol Maleate(IM) | Glycopyrrolate(GB) |
|---|---|---|
| MMAD (μm) | 3.28 | 2.93 |
| GSD (%) | 1.8 | 1.7 |

### Comparative Example 4

Indacaterol maleate and glycopyrrolate according to the amounts in Table 18, were dissolved in 20 g of 95% EtOH. EDTA and 50% BAC according to the amounts in Table 18 was then added to the solution followed by 70 ml of purified water and the resulting mixture sonicated until the components completely dissolved. The solution was adjusted to the target pH with hydrochloric acid. Finally, purified water was added to a final volume of 100 ml.

TABLE 18
Contents of Comparative Example 4

| Ingredients | Comparative Example 4 |
|---|---|
| GB | 26 mg |
| IM | 59 mg |
| 95% EtOH | 20 g |
| EDTA | 11 mg |
| 50% BAC | 20 mg |
| HCl | Adjust to pH 3.9 |
| Purified water | Added to 100 ml |

TABLE 19
Stability of Comparative Example 4
(Condition: 40° C. ± 2° C./75% ± 5% RH)

| | | | 40° C. | 40° C. | 40° C. |
|---|---|---|---|---|---|
| | | 0 day | l month | 2 Months | 3 Months |
| Content | GB % | 100.81 | 102.13 | 99.82 | 100.74 |
| | IM % | 100.8 | 99.73 | 94.7 | 96.72 |
| Impurities % | Maximum | ND | 2.07 | 2.07 | 5.74 |
| | impurity(%) | | | | |
| | Total | ND | 2.33 | 2.33 | 5.98 |
| | impurity(%) | | | | |

TABLE 20
Stability of Comparative Example 4
(Condition: 25° C. ± 2° C./75% ± 5% RH)

| | | | 25° C. | 25° C. | 25° C. |
|---|---|---|---|---|---|
| | | 0 day | l month | 2 Months | 3 Months |
| Content | GB % | 100.81 | 101.35 | 101.37 | 101.60 |
| | IM % | 100.8 | 101.35 | 101.37 | 101.60 |
| Impurities % | Maximum | ND | 0.23 | 0.56 | 3.35 |
| | impurity(%) | | | | |
| | Total | ND | 0.39 | 0.8 | 3.58 |
| | impurity(%) | | | | |

As can been seen from Tables 19 and 20, Comparative Example 4 is not stable at 40° C.±2° C./75%±5% RH and 25° C.±2° C./75%±5% RH. The total impurities of Comparative Example 4 reached 5.98% for 3 Months at 40° C.±2° C./75%±5% RH. The total impurities of Comparative Example 4 reached 3.58% for 3 Months at 25° C.±2° C./75%±5% RH. However, the total impurity of the invention is only 0.29% for 6 months at 40° C.±2° C./75%±5% RH. The formulation of the invention is much more stable than the formulation of Comparative Example 4.

### Comparative Example 5

Solubility comparison test:

WO2020019952A1 describes a formulation containing 45 mg of glycopyrronium and 90 mg of indacaterol maleate, which is equivalent to 56.7 mg of glycopyrronium bromide and 117 mg of indacaterol maleate. The contents of the formulation are provided in Table 21:

TABLE 21
Contents of Comparative Example 5

| Ingredients | Comparative Example 5 |
|---|---|
| glycopyrronium bromide | 56.7 mg |
| indacaterol maleate | 117 mg |
| EDTA | 10 mg |
| 50% BAC | 20 mg |
| 1N HCl | Adjust to pH 3.9 |
| Purified water | Added to 100 g |

The steps for making the formulation are as follows:
1. Weigh a 250 ml beaker (133.38 g tare).
2. Weigh the amount of EDTA and add it to the beaker.
3. Use a weighing cup to weigh the amount of 50% BAC, add it to the beaker, and rinse the beaker three times with a small amount of pure water each time.
4. Add pure water to a total weight of 231.39 g.
5. Use ultrasound to completely dissolve the EDTA.
6. Adjust the pH to 3.0 with 1N hydrochloric acid.
7. Weigh the amount of GB and IM and add them to the beaker.
8. Add pure water to a total weight of 233.38 g.
9. Seal with parafilm and stir on a magnetic stirrer.

Experimental Results:

After stirring overnight, the solution was in a suspended state and not completely dissolved, as shown in the FIG. 5.

### Example 9

Solving the technical problem of slow dissolution of IM:

In actual production, we need to prepare a high-concentration solution of API first and then dilute it. Generally, the volume of solvent used to prepare high-concentration API is 10% of the prescription amount. If IM is added directly to a large volume of water it will float on the surface of the solvent and cannot be dissolved.

We have shown that the addition of ethanol can increase the dissolution rate of IM. Ethanol, however, makes the solution unstable. The addition of other organic solvents can also impact the character of the solution. Accelerating the dissolution rate of IM without affecting the stability of the solution is a technical problem that needs to be solved.

After continuous exploration, we have found that the following preparation methods can solve this technical problem.

### Preparation of Example 9

Dissolve 25 g of SBECD, 50 mg of EDTA, and 100 mg of 50% BAC in 50 g of water to provide a solution, then add 1.6 g of IM to the solution and stir the resulting suspension in a water bath at 60° C. The IM dissolves in about 1 hour. Add 800 mg of GB and stir the resulting mixture to completely dissolve the GB followed by 400 g of purified water. Finally, add pure water to provide a total weight of 509 g.

The problem of slow dissolving can be solved by heating the suspension of IM.

TABLE 22
Contents of Example 9

| Ingredients | Example 9 |
|---|---|
| GB | 800 mg |
| IM | 1.6 g |
| SBECD | 25 g |
| EDTA | 50 mg |
| 50% BAC | 100 mg |
| 1N HCl | Adjust to pH 3 |
| Purified water | 509 g |

TABLE 23
Stability of Example 9 (Condition: 60° C. ± 2° C./75% ± 5% RH)

| | GB | | IM | |
|---|---|---|---|---|
| Sample name | mg/ml | content % | mg/ml | content % |
| 0 day | 1.605 | 100.29% | 3.207 | 100.21% |
| 60° C. 3 days | 1.624 | 101.53% | 3.240 | 101.24% |
| 60° C. 7 days | 1.677 | 104.82% | 3.332 | 104.14% |

TABLE 24
Stability of Example 9 (Condition: 60° C. ± 2° C./75% ± 5% RH)

| Impurities | RRT | Example 9-0 day | Example 9-60° C. 3 day | Example 9-60° C. 7 day |
|---|---|---|---|---|
| Unknown impurity | 0.641 | 0.01% | 0.02% | 0.01% |
| IM-10 | 1.046 | 0.07% | 0.07% | 0.06% |
| Unknown impurity | 1.219 | 0.02% | 0.02% | 0.02% |
| Unknown impurity | 1.435 | 0.01% | 0.01% | 0.01% |

| | | | |
|---|---|---|---|
| Maximum impurity | 0.07% | 0.07% | 0.06% |
| Total Impurities | 0.11% | 0.12% | 0.10% |

It can be seen from Tables 23 and 24 that the preparation method according to this example, which uses heat to accelerate the dissolution rate of IM, does not affect the stability of the resulting formulation.

Therefore, heating the solution when dissolving IM is an effective measure to solve the slow dissolution rate of IM.

### Comparative Example 6

Comparison of atomization with different devices:

The atomization of two devices were compared: 1. A soft mist inhaler and 2. An atomization device.
1. The soft mist inhaler is the device depicted in FIG. 1.
2. The atomization device is the Ulyibro® breezhaler®, purchased from Novartis.

Administration using a soft mist inhalation device

TABLE 25
Contents of Sample 17

| Ingredients | Sample 17 |
|---|---|
| IM | 320 mg |
| GB | 160 mg |
| SBECD | 5 g |
| 50% BAC | 20 mg |
| EDTA | 10 mg |
| 1N HCl | Adjusted to pH |
| | 3.0 |
| Purified water | Added to 101.8 g |

The preparation of Sample 17 inhalation solution:

EDTA, 50% BAC, and SBECD according to the amounts in Table 25 were dissolved in 98 g of purified water and the resulting solution adjusted to the target pH with HCl. GB and IM according to the amounts in Table 25 were added to the solution, and the resulting mixture sonicated until the components completely dissolved. Finally, purified water was added to provide a final weight of 101.8 g.

The aerodynamic particle size distribution was then determined using a Next Generation Impactor (NGI) instrument. The soft mist inhaler was held close to the NGI inlet until no aerosol was visible. The flow rate of the NGI was set to 30 L/minute and was operated at ambient temperature and a relative humidity (RH) of 90±2%.

Sample 17 was discharged into the NGI. Fractions of the dose were deposited at different stages of the NGI, in accordance with the particle size of the fraction. Each fraction was washed from the stage and analyzed using HPLC.

The results are provided in Table 26 below.

TABLE 26
Aerodynamic Particle Size Distribution of GB of Sample 17

| | | Percentage content | Cut-off |
|---|---|---|---|
| | Dosage (μg) | at all levels | diameter (μm) |
| Throat | 3.95 | 11.17% | |
| Stage 1 | 1.39 | 3.93% | 11.76 |
| Stage 2 | 6.21 | 17.56% | 6.40 |
| Stage 3 | 8.5 | 24.04% | 3.99 |
| Stage 4 | 8.58 | 24.26% | 2.30 |
| Stage 5 | 4.07 | 11.51% | 1.36 |
| Stage 6 | 1.38 | 3.90% | 0.83 |
| Stage 7 | 0.86 | 2.43% | 0.54 |
| MOC | 0.42 | 1.19% | 0.36 |

| | |
|---|---|
| Actual dose | 35.36 μg |
| The theory of dose | 35.36 μg |
| Recovery | 100.00% |
| FPF | 67.33% |
| ISM | 30.02 μg |

TABLE 27
Aerodynamic Particle Size Distribution of IM of Sample 17

| | IM of Sample 17 | | Cut-off |
|---|---|---|---|
| | | Percentage content at all | diameter |
| IM Deposited | Dosage (μg) | levels | (μm) |
| Throat | 7.49 | 10.86% | |
| Stage 1 | 2.43 | 3.52% | 11.76 |
| Stage 2 | 12.46 | 18.07% | 6.40 |
| Stage 3 | 17.18 | 24.92% | 3.99 |
| Stage 4 | 17.44 | 25.30% | 2.30 |
| Stage 5 | 7.73 | 11.21% | 1.36 |
| Stage 6 | 2.64 | 3.83% | 0.83 |
| Stage 7 | 1.18 | 1.71% | 0.54 |
| MOC | 0.39 | 0.57% | 0.36 |

| | |
|---|---|
| Actual dose | 68.94 μg |
| The theory of dose | 70.72 μg |
| Recovery | 97.48% |
| FPF | 67.54% |
| ISM | 59.02 μg |

ISM is Impactor Size Mass.
MOC is Micro-Orifice Collector.
FPF (Fine Particle Fraction) is the proportion of fine particle dose in the released dose.
$FPF = \frac{Mass < 5 μm}{Mass Total dose} .$ The larger the FPF value, the higher the lung deposition efficiency.

Administration using an atomization device.

The sample is a GB and IM-containing dry powder capsule, purchased from Novartis Europharm limited, United Kingdom. The device used to administer the dry powder was a Ulyibro® breezhaler®, purchased from Novartis.

The dry power capsule was discharged into the NGI. Fractions of the dose were deposited at different stages of the NGI, in accordance with the particle size of the fraction. Each fraction was washed from the stage and analyzed using HPLC. The results are provided below in Table 28.

TABLE 28
Aerodynamic Particle Size Distribution of GB of Dry Powder Capsule

| | Dosage | Percentage content | Cut-off |
|---|---|---|---|
| GB Deposited | (μg) | at all levels | diameter (μm) |
| Capsule | 5.08 | 8.21% | |
| Device | 10.47 | 16.93% | |
| Throat | 5.42 | 8.76% | |
| Stage 1 | 7.83 | 12.66% | 11.76 |
| Stage 2 | 0.8 | 1.29% | 6.40 |
| Stage 3 | 1.67 | 2.70% | 3.99 |
| Stage 4 | 6.14 | 9.93% | 2.30 |
| Stage 5 | 15.22 | 24.61% | 1.36 |
| Stage 6 | 7.1 | 11.48% | 0.83 |
| Stage 7 | 2.11 | 3.41% | 0.54 |
| Micro-Orifice Collector | 0 | 0.00% | 0.36 |
| (MOC) | | | |
| Theoretical dose | 63 μg | | |
| Actual test dose | 61.84 μg | | |
| Recovery rate | 98.16% | | |
| Fine Particle Fraction | 52.13% | | |
| (FPF) | | | |
| ISM | 32.24 μg | | |

TABLE 29
Aerodynamic Particle Size Distribution of IM of Dry Powder Capsule

| | Dosage | Percentage content | Cut-off |
|---|---|---|---|
| IM Deposited | (μg) | at all levels | diameter (μm) |
| Capsule | 6.92 | 4.89% | |
| Device | 30.55 | 21.57% | |
| Throat | 16.86 | 11.90% | |
| Stage 1 | 26.39 | 18.63% | 11.76 |
| Stage 2 | 2.35 | 1.66% | 6.40 |
| Stage 3 | 5.98 | 4.22% | 3.99 |
| Stage 4 | 13.22 | 9.33% | 2.30 |
| Stage 5 | 23.75 | 16.77% | 1.36 |
| Stage 6 | 11.61 | 8.20% | 0.83 |
| Stage 7 | 3.19 | 2.25% | 0.54 |
| Micro-Orifice Collector | 0.73 | 0.52% | 0.36 |
| (MOC) | | | |
| Theoretical dose | 143 μg | | |
| Actual test dose | 141.55 μg | | |
| Recovery rate | 99.05% | | |
| Fine Particle Fraction | 41.29% | | |
| (FPF) | | | |
| ISM | 58.57 μg | | |

Table 24 shows that the fine particle fraction (FPF) is only 52.13%, which is far lower than the FPF value using the soft mist inhaler. When the atomization device purchased from Novartis is used to atomize the GB+IM-containing dry power capsule a large amount of drug remains inside the capsule, the device, and the simulated throat. The drug remaining inside the capsule, the device, and the throat cannot reach the lungs to provide a therapeutic effect. The experimental results show that the formulation of the present invention administered using a soft mist device is atomized more effectively than when administered as a dry power using an Ulyibro® breezhaler® device. Therefore, the daily dose of GB and IM administered using a formulation according to the invention with a soft mist inhaler is about 35 μg and about 70 μg, respectively. The daily dose for administration as dry power is 63 μg of GB and 143 μg of IM. From the point of daily dose, the dose of GB and IM administered using a formulation according to the invention is about half of the dose administered using a dry powder formulation, while achieving the same therapeutic effect as the dry powder. A lower dose can reduce the side effects of drugs on the human body.

What is claimed is:

1. A liquid, propellant-free pharmaceutical formulation comprising:
(a) glycopyrrolate and indacaterol maleate;
(b) a solvent;
(c) a pharmacologically acceptable solubilizing agent; and
(d) a pharmacologically acceptable preservative,
wherein the pharmaceutical formulation has a pH ranging from about 2.0 to about 6.0.

2. The pharmaceutical formulation according to claim 1, wherein glycopyrrolate is present in an amount ranging from about 0.2 mg/100 ml to about 550 mg/100 ml.

3. The pharmaceutical formulation according to claim 1, wherein the indacaterol maleate is present in an amount ranging from about 0.34 mg/100 ml to about 1000 mg/100 ml.

4. The pharmaceutical formulation according to claim 1, wherein the solvent is a water substantially free of other solvents.

5. The pharmaceutical formulation according to claim 1, wherein the solubilizing agent is selected from the group consisting of tween-80, poloxamer, polyoxyethylated castor oil, polyethylene glycol, solutol HS 15, polyvinylpyrrolidone, cyclodextrin derivatives, sulfobutylether β-cyclodextrin, and combinations thereof.

6. The pharmaceutical formulation according to claim 5, wherein the solubilizing agent is present in an amount ranging from about 1 g/100 ml to about 40 g/100 ml.

7. The pharmaceutical formulation according to claim 1, wherein the pharmacologically acceptable preservative is selected from the group consisting of benzalkonium chloride, benzoic acid, sodium benzoate, and combinations thereof.

8. The pharmaceutical formulation according to claim 7, wherein the preservative is present in an amount ranging from about 2 mg/100 ml to about 300 mg/100 ml.

9. The pharmaceutical formulation according to claim 1, wherein the stabilizer is selected from the group consisting of edetic acid, edetate disodium dehydrate, edetate disodium, citric acid, and combinations thereof.

10. The pharmaceutical formulation according to claim 9, wherein the stabilizer is present in an amount ranging from about 1 mg/100 ml to about 500 mg/100 ml.

11. The pharmaceutical formulation according to claim 1, wherein the pharmaceutical formulation comprises a pharmacologically acceptable additive.

12. The pharmaceutical formulation according to claim 11, wherein pharmacologically acceptable additive is an antioxidant.

13. A method for administering the pharmaceutical formulation according to claim 1, comprising nebulizing the pharmaceutical formulation using an inhaler depicted in FIG. 1.

14. A method for administering the pharmaceutical formulation according to claim 1, comprising nebulizing a defined amount of the pharmaceutical formulation with an inhaler by using pressure to force the pharmaceutical formulation through a nozzle to form an inhalable aerosol.

15. The method according to claim 14, wherein the defined amount of the pharmaceutical formulation ranges from about 5 microliters to about 30 microliters.

16. The method according to claim 14, wherein aerosol has an MMAD of less than about 10 μm.

17. The method according to claim 14, wherein aerosol has a D50 of less than about 10 μm.

18. The method according to claim 13, wherein the inhaler further comprises a block function and a counter.

19. A method of treating asthma or COPD in a patient, comprising administering to the patient the pharmaceutical formulation according to claim 1.

20. The method of claim 19, wherein the pharmaceutical formulation is administered at a therapeutically effective dose of glycopyrronium bromide ranging from about 1 μg to about 142 μg and a therapeutically effective dose of indacaterol maleate ranging from about 5 μg to about 500 μg.

21. A liquid, propellant-free pharmaceutical formulation comprising:
an aqueous solution of:
(a) glycopyrrolate in an amount of about 0.2 mg/100 mL to about 550 mg/mL,
(b) indacaterol maleate in an amount of about 0.3 mg/100 mL to about 1000 mg/100 mL,
(c) sulfobutylether β-cyclodextrin (SBECD) in an amount of about 1 g/100 mL to about 40 g/mL,
(d) 50% benzalkonium chloride aqueous solution in an amount of about 2 mg/100 mL to about 300 mg/mL, and
(e) edetate disodium dihydrate in an amount of about 1 mg/100 mL to about 500 mg/mL,
wherein the pharmaceutical preparation has a pH ranging from about 3.0 to about 4.0.

22. A method for preparing a pharmaceutical formulation comprising a solution of indacaterol maleate in water comprising: (i) adding about 3.4 mg to about 10 g of indacaterol maleate to about 100 g of water substantially free of other solvents to provide a suspension and (ii) heating the suspension at a temperature of at least about 50° C. to about 90° C. with stirring until the indacaterol dissolves.

23. The method of claim 22 comprising:
(i) combining about 10 g to about 400 g of sulfobutylether β-cyclodextrin (SBECD), about 10 mg to about 5000 mg of EDTA, about 20 mg to about 3000 mg of 50% benzalkonium chloride aqueous solution, and about 50 g of water to provide a first solution;
(ii) adding about 3.4 mg to about 10 g of indacaterol maleate to about 50 g of water to provide a first suspension;
(iii) combining the first suspension with the first solution to provide a second suspension;
(iv) heating the second suspension at a temperature of about 50° C. to about 90° C. with stirring until the indacaterol maleate dissolves to provide a second solution;
(v) adding about 20 mg to about 3000 mg of glycopyrronium bromide to the second solution to provide a mixture and stirring the mixture until the glycopyrronium bromide dissolves.
